(19) United States
(12) Patent Application Publication
(10) Pub. No.: US 2011/0042655 A1
(43) Pub. Date: Feb. 24, 2011

(54) AROMATIC ELECTROLUMINESCENT COMPOUNDS WITH HIGH EFFICIENCY AND ELECTROLUMINESCENT DEVICE USING THE SAME

(75) Inventors: Sung-Min Kim, Seoul (KR); Bong Ok Kim, Seoul (KR); Mi Young Kwak, Seoul (KR); Seung Soo Yoon, Seoul (KR)

Correspondence Address: ROHM AND HAAS ELECTRONIC MATERIALS LLC, 455 FOREST STREET, MARLBOROUGH, MA, 01752, US

(73) Assignee: Gracel Display Inc., Seongdong-gu, Seoul (KR)

(21) Appl. No.: 12/743,190

(22) PCT Filed: Nov. 22, 2007
(86) PCT No.: PCT/KR07/05911

§ 371 Date: Oct. 29, 2010

(51) Int. Cl. H01L 51/54; C07C 13/66; C07C 13/62

(52) U.S. Cl. 257/40; 585/26; 257/E51.041

(57) ABSTRACT

The present invention relates to an organic electroluminescent compound including a fusion ring and an organic elecotroluminescent including the same. The organic electroluminescent according to the present invention has an advantage of exhibiting an EL properties superior to existing electroluminescent materials as it has a good thin film stability due to a low crystallization and a satisfactory color purity.

### TECHNICAL FIELD

The present invention relates to electroluminescent compounds including a fusion ring and electroluminescent device using the same

### BACKGROUND ART

Recently, the rapid coming of the information age has increased an importance of a display which serves as an interface between human beings and electronic information devices. An organic light emitting device (OLED) has been actively studied in the world as a new flat panel display technology, and this is because the OLED is of a self-luminous type, has a superior display property and can be easily manufactured as it has a simple device structure and it is possible to manufacture an ultrathin and ultralight display using it. The OLED device is generally includes a thin layer of various organic compounds between a cathode and an anode which are made of metal, electrons and holes injected through the cathode and the anode are transported respectively through an electron injection layer and an electron transport layer, and a hole injection layer and a hole transport layer to an electroluminescent layer to form an exciton, and the formed exciton is disintegrated in stable state to emit light. At this time, as the properties of the OLED device is highly dependent upon properties of the used organic luminescent compound, studies for luminescent materials are actively carried out.

The luminescent materials can be divided in a functional aspect into a host material and a dopant material and a device structure with the most superior electroluminescent property has been generally known that its electroluminescent layer is manufactured by doping a dopant into a host. Recently, development of an organic electroluminescent (EL) device with high efficiency and long life is urgently required and development of a material which is much superior to existing electroluminescent material is urgently required particularly in consideration of an EL property level required from a medium or large size OLED panel. In this aspect, development of a host material is one of the important problems to solve. At this time, the host material which serves as a transporter of energy and a solvent in a solid state in an organic EL device should have desirable properties that its degree of purity is high and it has proper molecular weight so as to permit a vapor deposition. In addition, it should have a high glass transition temperature and a high thermal decomposition temperature so as to obtain thermal stability it is required to have high electrochemical stability for long life it should be easy to form an amorphous thin film and it should have a superior adhesive force to materials in other adjacent layers whereas an interlayer movement should not occur.

A variety of host materials have been presented and a typical examples include diphenylvinyl-biphenyl (DPVBi) available from Idemitsu Kosan Co., Ltd. and dinaphthyl anthracene (DNA) available from Eastman Kodak Company, however there is much room for improvement in an efficiency, a life and a color purity.

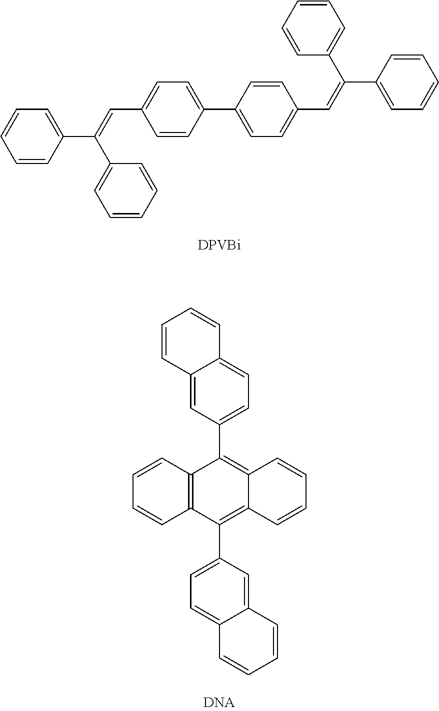

The DPVBi has a problem of a thermal stability as it has a glass transition temperature of below 100° C. and, in order to improve the problem, DPVPAN and DPVPBAN, in which anthracene and dianthracene are respectively introduced inside the biphenyl of the DPVBi, have been developed to increase the glass transition temperature to more than 105° C. thereby improving the thermal stability, however the color purity and a luminescent efficiency have not reached to a satisfactory level.

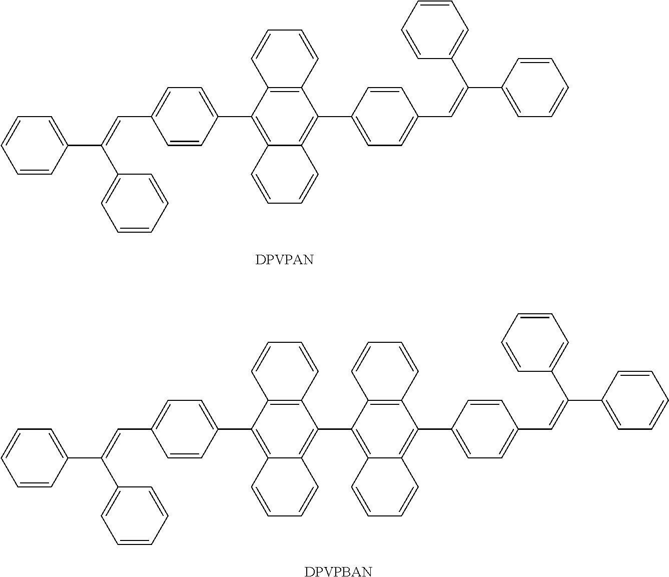

In addition, in the DNA has found a phenomenon that it has low thin film stability thus is easily crystallized from the result of observing the thin film formed on an ITO through a vapor deposition using a scanning probe microscopy. The phenomenon has been known to have a bad influence on the life of the device and in order to improve this shortcoming of the DNA, mDNA and tBDNA, in which methyl group or t-butyl group are introduced into a second position of the DNA, have been developed to destroy the symmetry of a molecule thereby improving the film stability, however the color purity and the electroluminescent efficiency have not reached to a satisfactory level, too.

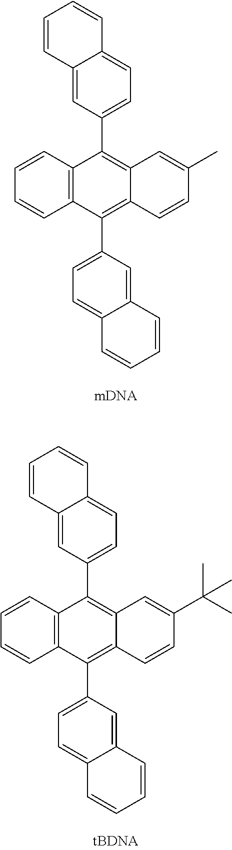

### DISCLOSURE

### [Technical Problem]

It is an object of the present invention to provide an organic electroluminescent compound with a superior skeleton, which has a luminescent efficiency superior to existing host materials and a proper color coordinates and to provide an organic electroluminescent compound having good thin film stability due to a low crystallization. It is another object of the present invention to provide an electroluminescent device using the above organic electroluminescent compound.

### [Technical Solution]

Hereinafter, the present invention is described in detail.

The present invention relates to an organic electroluminescent compound including a fusion ring represented by the following formula 1 and an organic light emitting diode (OLED) using the same as an electroluminescent material. The organic electroluminescent compound of the present invention also is used as other layers as well as luminescent layer.

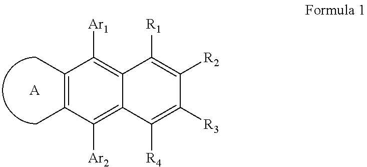

wherein, a ring A is a fused aryl group in which at least two rings are fused; Ar₁ and Ar₂ are independently a C₆-C₃₀ aryl group R₁ to R₄ are independently a hydrogen, a C₁-C₂₀ straight or branched chain alkyl group or alkoxy group and a C₆-C₃₀ aryl or heteroaryl group and a halogen group; and the fused aryl group, the aryl group, heteroaryl group, the alkyl group and the alkoxy group are optionally substituted by a C₁-C₂₀ straight or branched chain alkyl group, a aryl group and halogen group.

The organic electroluminescent compound according to the present invention is characterized in that, in the formula 1, the ring A forms at least two fusion rings, and can be specifically represented by following formula 2 to formula 7:

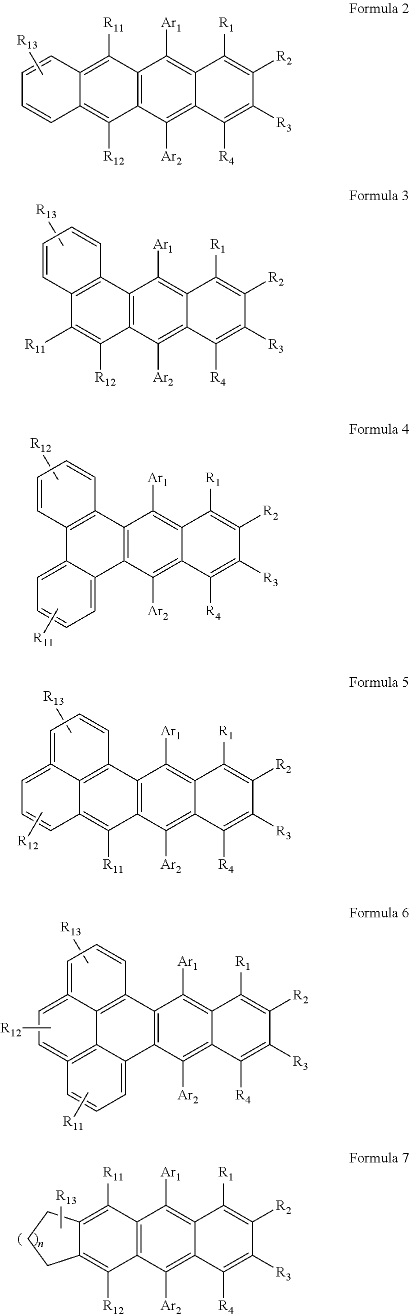

wherein, in the formula 2 to formula 7, Ar₁, Ar₂, R₁, R₂, R₃ and R₄ are the same as those defined in the formula 1 and R₁₁ to R₁₃ are independently a hydrogen, a C₁-C₂₀ straight or branched chain alkyl group or alkoxy group and a C₆-C₃₀ aryl or heteroaryl group and a halogen group; n is 1 to 3; and the alkyl group and the alkoxy group, the aryl group and heteroaryl group are optionally substituted by a C₁-C₂₀ straight or branched chain alkyl group, a aryl group and halogen group.

In the above formula 1 to formula 7, the Ar₁ and Ar₂ may be independently phenyl, tolyl, biphenyl, naphthyl, anthryl and fluorenyl, and the R₁ to R₄ and R₁₁ to R₁₃ include independently hydrogen, methyl, ethyl, propyl, isopropyl, butyl, isobutyl, pentyl, hexyl, ethylhexyl, heptyl, octyl, isooctyl, nonyl, decyl, dodecyl, hexadecyl, cyclopentyl, cyclohexyl, phenyl, tolyl, byphenyl, benzyl, naphthyl, anthryl and fluorenyl.

The organic electroluminescent compound according to the present invention may be, but not limited to, the following compounds:

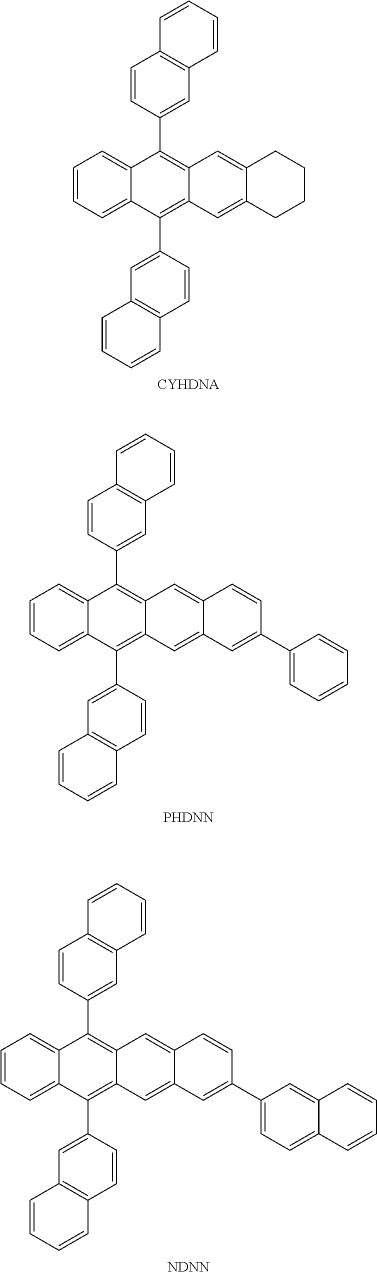

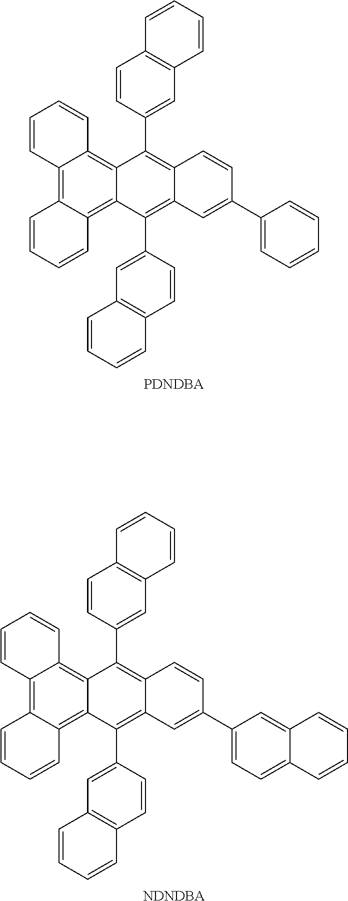

### MODE FOR INVENTION

### PREPARATION EXAMPLE 1

### Preparation of CYHDNA

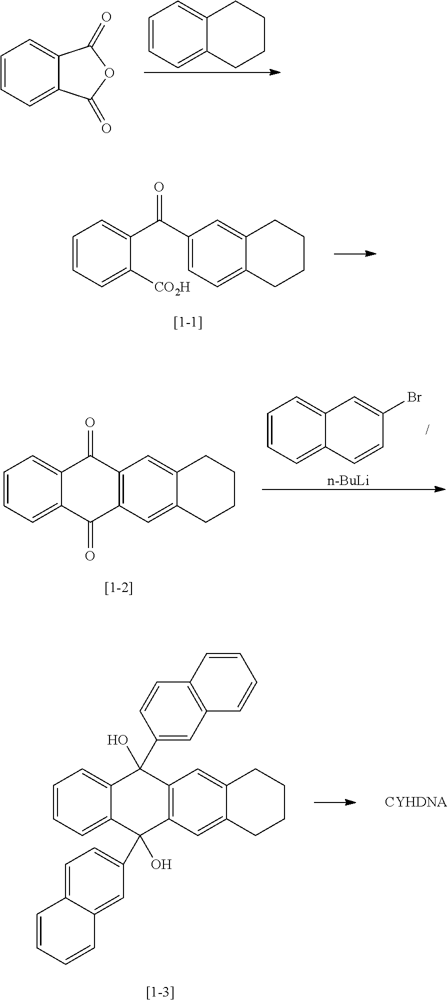

Into a round bottom flask were put 70 ml of dichloromethane and 15.8 g (118.8 mmol) of aluminum chloride and then 8.0 g (54.0 mmol) of isobenzofuran-1,3-dione and 8.8 mL (64.8 mmol) of 1,2,3,4-tetrahydronaphthalene were dissolved in 800 mL of dichloromethane and added slowly to the flask filled with the aluminum chloride. After stirring at 25° C. for 24 hours, the reaction mixture were added slowly to mixture solution of 30 mL of 35% hydrochloric acid and 150 mL of ice water and further stirred for 20 minutes. The reaction mixture was extracted using 200 mL of ethylacetate, recrystallized and then dried thereby obtaining 10.6 g (37.8 mmol) of a compound [1-1].

10.6 g (37.8 mmol) of the compound [1-1], 50.4 g (378.1 mmol) of aluminum chloride and 11.1 g (189.0 mmol) of sodium chloride were put and reflux stirred at 130° C. for 4 hours. The reaction product were cooled to 25° C. and then added with and dissolved in 60 mL of tetrahydrofuran and added with 30 ml of water, thereby completing the reaction. After the reaction was completed, the reaction product was extracted with 100 mL of dichloromethane and dried under reduced pressure thereby obtaining 3 g (11.4 mmol) of a compound [1-2].

After dissolving 8.5 g (40.9 mmol) of 2-bromo naphthalene in 50 mL of tetrahydrofuran, 4.3 mL (45.7 mmol) of n-buthyllithium (2.5 M solution in n-Hexane) was added slowly to 50 mL of the tetrahydrofuran dissolved with the 2-bromonaphthalene at −72° C. and then stirred for 2 hours and then 3.0 g (11.4 mmol) of the compound [1-2] was added thereto and stirred at a room temperature for 24 hours. After completing reaction by adding slowly 50 mL of distilled water, the reaction mixture was extracted with 250 mL of tetrahydrofuran and dried under reduced pressure thereby obtaining 3.5 g (6.8 mmol) of a compound [1-3].

3.5 g (6.8 mmol) of the compound [1-3], 4.5 g (27.1 mmol) of potassium iodide and 5.8 g (54.6 mmol) of sodium hydrophosphinate were dissolved in a mixture solution of 30 mL of acetic acid and 10 ml of dichloromethane and reflux stirred for 24 hours. After completing reaction by cooling the reaction product to 25° C. and adding slowly 20 mL of water thereto, the reaction product was extracted with 200 mL of dichloromethane, recrystallized and dried thereby obtaining 2.8 g (5.8 mmol) of a compound CYHDNA with a total yield of 11%.

¹H NMR (200 MHz, CDCl₃): δ=1.60 (m, 4H), 2.85 (m, 4H), 7.32 (m, 6H), 7.40 (t, 2H), 7.54 (d, 2H), 7.67-7.73 (m, 8H), 7.89 (d, 2H)

MS/FAB: 484.22 (found), 484.63 (calculated)

### PREPARATION EXAMPLE 2

### Preparation of PHDNN

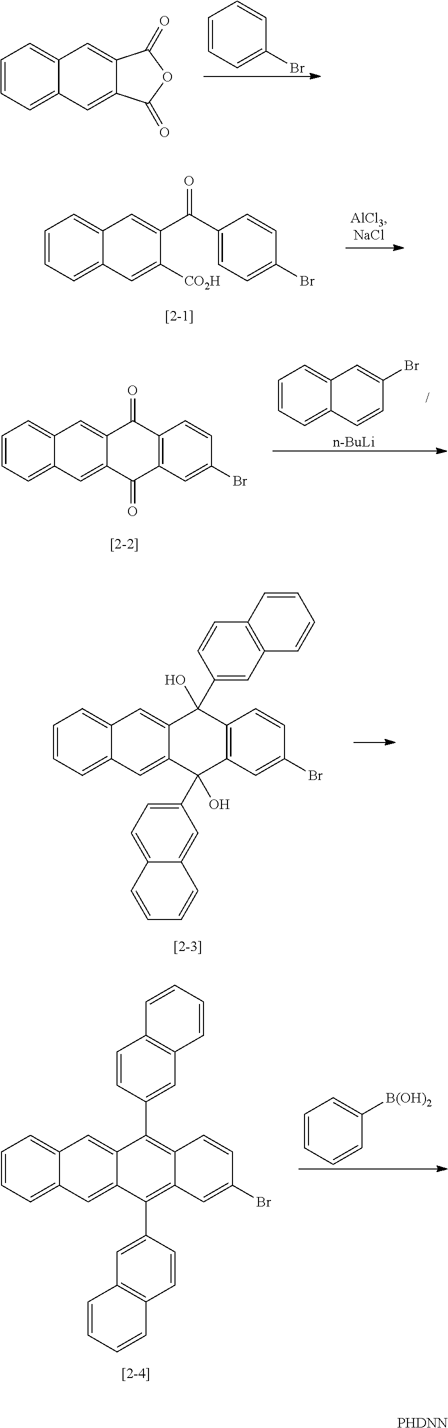

Using 10 g (50.5 mmol) of naphtho(2,3-C)furan-1,3-dione and 9.5 g (60.5 mmol) of 1-bromobenzene, 12.5 g (35.2 mmol) of a compound [2-1] was obtained by a manner similar to the Preparation Example 1.

Using 12.5 g (35.2 mmol) of the compound [2-1], 46.9 g (351.9 mmol) of aluminum chloride and 10.3 g (175.9 mmol) of sodium chloride, 3.6 g (10.6 mmol) of a compound [2-2] was obtained by the same manner as in the Preparation Example 1.

Using 8.0 g (38.6 mmol) of 2-bromonaphthalene, 3.9 mL(42.7 mmol) of n-butyllithium (2.5 M solution in n-hexane) and 3.6 g (10.6 mmol) of the compound [2-2], 3.8 g (6.4 mmol) of a compound [2-3] was obtained by a manner similar to the Preparation Example 1.

Using 3.8 g (6.4 mmol) of the compound [2-3], 4.2 g (25.3 mmol) of potassium iodide and 5.4 g (50.9 mmol) of sodium hydrophosphinate, 2.9 g (5.2 mmol) of a compound [2-4] was obtained by the manner of Preparation Example 1.

2.9 g (5.2 mmol) of the compound [2-4] and 0.7 g (6.0 mmol) of phenylboronic acid were dissolved in a mixture solution of 30 mL of toluene and 15 ml of ethanol and added with 0.2 g (1.7 mmol) of tetrakis(triphenylphosphine) palladium(O) [Pd(PPh₃)₄] and 2.3 mL of 2 M sodium carbonate aqueous solution and then reflux stirred for 5 hours. After completing reaction by cooling the reaction product to a room temperature and adding slowly 15 mL of water thereto, the reaction mixture was extracted with 300 mL of dichloromethane and dried under reduced pressure thereby obtaining 2.6 g (4.7 mmol) of a compound PHDNN with a total yield of 9%.

¹H NMR (200 MHz, CDCl₃): δ=7.22-7.32 (m, 9H), 7.48 (d, 2H), 7.54 (d, 3H), 7.67-7.73 (m, 11H), 7.89 (d, 3H)

MS/FAB: 556.22 (found), 556.69 (calculated)

### PREPARATION EXAMPLE 3

### Preparation of NDNN

3.0 g (4.9 mmol) of a compound NDNN was obtained with a total yield of 9% by the same manner as in the Preparation Example 2 except for using 2.9 g (5.2 mmol) of the compound [2-4] and 1.1 g (6.4 mmol) of naphthaleneboronic acid.

¹H NMR (200 MHz, CDCl₃): δ=7.32 (m, 8H), 7.54 (d, 4H), 7.67-7.73 (m, 14H), 7.89 (d, 4H)

MS/FAB: 606.23 (found), 606.75 (calculated)

### PREPARATION EXAMPLE 4

### Preparation of PDNBA

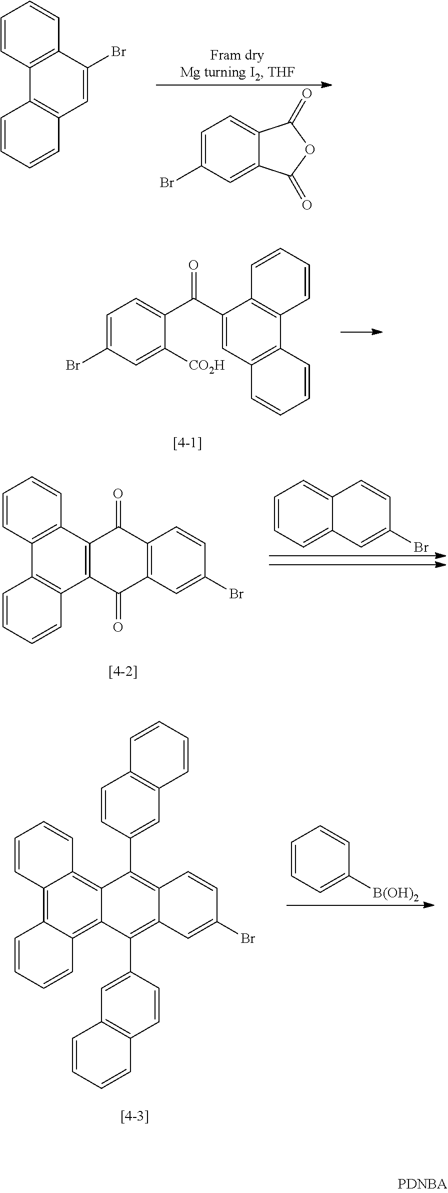

Into a round bottom flask (100 ml) was put 1.7 g (70.1 mmol) of Mg turning and then were put small amount of I₂ pieces and 10 ml of tetrahydrofuran. 11 g (42.5 mmol) of 9-bromophenanthrene was dissolved in 10 mL of tetrahydrofuran and added slowly to the flask filled with the magnesium of 0° C. and then stirred at 25° C. for 30 minutes. 9.9 g (43.4 mmol) of 5-bromoisobenzofuran-1,3-dione and 12.7 g (95.6 mmol) of aluminum chloride were put into the flask and stirred for 24 hours. After adding the reaction solution slowly to 150 mL of 1N hydrochloric acid aqueous solution and stirring for 30 minutes, the reaction solution was extracted with 200 mL of dichloromethane and dried under reduced pressure, thereby obtaining 11.4 g (28.2 mmol) of a compound [4-1].

Using 11.4 g (28.2 mmol) of the compound [4-1], 37.9 g (284.4 mmol) of aluminum chloride and 8.3 g (142.2 mmol) of sodium chloride, 2.6 g (6.8 mmol) of a compound [4-2] was obtained by the manner of the Preparation Example 2.

Using 5.1 g (24.6 mmol) of 2-bromonaphthalene, 2.5 mL(27.3 mmol) of n-butyllithium (2.5 M solution in n-hexane) and 2.6 g (6.8 mmol) of the compound [4-2], 2.2 g (3.7 mmol) of a dihydroxy compound was obtained by the manner of the Preparation Example 2. Using 2.2 g (3.7 mmol) of the dihydroxy compound, 2.5 g (14.8 mmol) of potassium iodide and 3.1 g (29.6 mmol) of sodium hydrophosphinate, 1.95 g (3.2 mmol) of a compound [4⁻3] was obtained by the manner of the Preparation Example 3.

Using 1.95 g (3.2 mmol) of the compound [4-3], 470.7 mg (3.9 mmol) of phenylboronic acid, 0.2 g (1.7 mmol) of tetrakis(triphenylphosphine)palladium(O) [Pd(PPh₃)₄] and 2.3 mL of 2 M sodium carbonate aqueous solution, 1.16 g (2.3 mmol) of a compound PDNBA was obtained with a total yield of 5% by the same manner as in the Preparation Example 3.

¹H NMR (200 MHz, CDCl₃): δ=7.22-7.32 (m, 9H), 7.48-7.54 (m, 7H), 7.73 (d, 1H), 7.82-7.89 (d, 2H), 8.93 (d, 2H)

MS/FAB: 506.2 (found), 506.63 (calculated)

### PREPARATION EXAMPLE 5

### Preparation of NDNDBA

using 1.95 g (3.2 mmol) of the compound [4-3] prepared in the Preparation Example 4, 0.664 g (3.9 mmol) of naphthaleneboronic acid, 0.2 g (1.7 mmol) of tetrakis(triphenylphosphine)palladium(O) [Pd(PPh₃)₄], 2.3 ml of 2 M sodium carbonate aqueous solution and a mixture solution of 30 mL of toluene and 15 mL of ethanol, 1.2 g (2.2 mmol) of a compound NDNDBA was obtained with a total yield of 5% by the same manner as in the Preparation Example 2.

¹H NMR (200 MHz, CDCl₃): δ=7.22-7.32 (m, 8H), 7.48-7.54 (m, 6H), 7.67-7.89 (m, 10H), 8.12 (d, 2H), 8.93 (d, 2H)

MS/FAB: 556.22 (found), 556.69 (calculated)

### EXAMPLE 1

### Manufacture of OLED Device Using the Compound According to the Present Invention

An OLED device with a structure using the electroluminescent material of the present invention was manufactured.

Firstly, a thin film of a transparent electrode indium-tin oxide(ITO) (15Ω/□) obtained from a glass for OLED was used after cleaned with ultrasonic wave using sequentially trichloroethylene, acetone, ethanol and distilled water and the put into isopropanol to store.

Next, The ITO substrate was loaded on a substrate folder of a vapor deposition equipment and 4,4′,4″-tris(N,N-(2-naphthyl)-phenylamino)triphenylamine (2-TNATA) having the following structure was put into a cell of the vapor deposition equipment and exhaustion was carried out until the degree of vacuum in a chamber reaches to 10⁻⁶ torr and then current was applied to the cell to vaporize 2-TNATA, thereby vapor depositing a hole injection layer having a thickness of 60 nm on the ITO substrate.

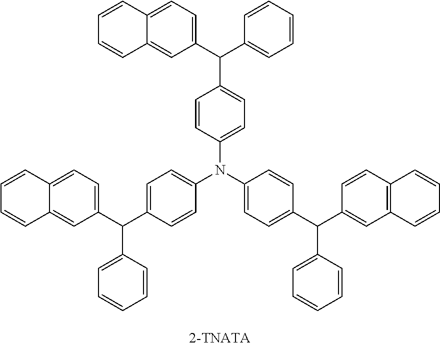

Subsequently, N′-bis(α-naphthyl)-N,N′-diphenyl-4,4′-diamine (NPB) having the following structure was put into the other cell in the vapor deposition equipment and then current was applied to the cell to vaporize NBP, thereby vapor depositing a hole transport layer having a thickness of 20 nm on the hole injection layer.

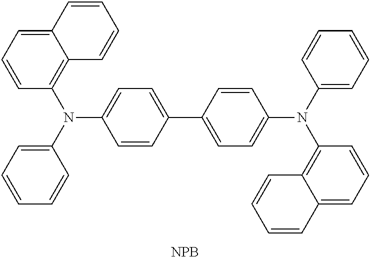

After forming the hole injection layer and the hole transport layer, an electroluminescent layer was vapor deposited thereon as follows. A compound (for example, the compound CYHDNA) according to the present invention was put into one cell of the vapor deposition equipment and a dopant electroluminescent material having the following structure was put into the other cell of the vapor deposition equipment, and then the electroluminescent layer having a thickness of 35 nm was vapor deposited at a deposition speed of 100:1 on the hole transport layer.

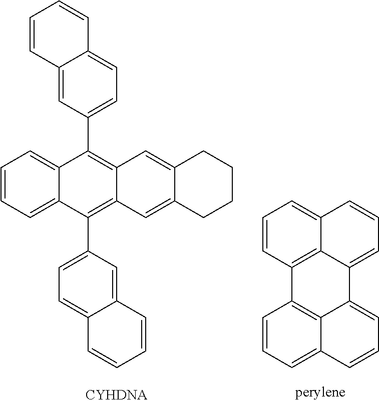

Subsequently, tris(8-hydroxyquinoline)aluminum(III) (Alq) having the following structure was deposited in a thickness of 20 nm as an electron transport layer, and then lithium quinolate (Liq) having the following structure was deposited in a thickness of 1 to 2 nm as an electron injection layer, and after that Al cathode was deposited in a thickness of 150 nm using another vapor deposition equipment, thereby manufacturing an OLED.

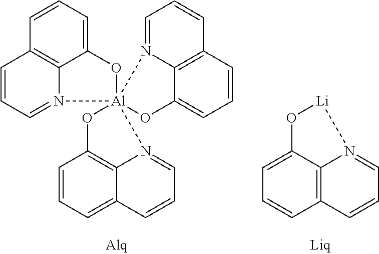

Each of the material used in the OLED device was purified by vacuum sublimation under 10⁻⁶ torr and then used as the electroluminescent material.

### COMPARATIVE EXAMPLE 1

### Manufacture of an OLED Device Using Conventional Electroluminescent Material

After forming the hole injection layer and the hole transport layer by the same manner as in the Example 1, dinaphthylanthracene (DNA) which is an electroluminescent for blue color was put into one cell of the vapor deposition equipment and perylene, which is another electroluminescent for blue color and has the following structure, was put into the other cell of the vapor deposition equipment, and then the electroluminescent layer having a thickness of 35 nm was vapor deposited at a deposition speed of 100:1 on the hole transport layer.

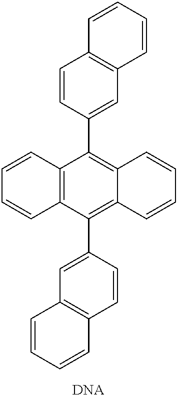

Subsequently, the electron transport layer and the electron injection layer were deposited by the same manner as in the Example 1 and then Al cathode was deposited in a thickness of 150 nm using another vapor deposition equipment, thereby manufacturing an OLED.

### EXAMPLE 2

### Electroluminescent Property of the Manufactured OLED Device

Electroluminescent efficiencies of OLED devices including the organic electroluminescent compound according to the present invention and conventional electroluminescent compound manufactured in the Example 1 and the Comparative Example 1 were measured at 500 cd/m² and 2,000 cd/m² respectively and shown in following Table 1. Particularly in an electroluminescent material for blue color, the measurement was carried out on a basis of luminance data in about 2,000 cd/m² since electroluminescent properties in a low luminance area and a luminance which is applied in a panel are very important.

TABLE 1

| | Electroluminescent | | | Electroluminescent | | Color | | Electrolum |
|---|---|---|---|---|---|---|---|---|
| | material | | EL peak | Efficiency(cd/A) | | coordinates | | inescent |
| No. | 1 | 2 | (nm) | @500 cd/m² | @2,000 cd/m² | X | Y | Efficiency/Y |
| 1 | CYHDNA | perylene | 460,488 | 5.23 | 4.77 | 0.162 | 0.212 | 22.5 |
| 2 | PHDNN | perylene | 464,492 | 6.23 | 5.66 | 0.165 | 0.223 | 25.4 |
| 3 | NDNN | perylene | 464,492 | 6.12 | 5.60 | 0.165 | 0.225 | 24.9 |
| 4 | PDNDBA | perylene | 466,496 | 6.69 | 5.92 | 0.164 | 0.237 | 25.0 |
| 5 | NDNDBA | perylene | 466,498 | 6.70 | 5.81 | 0.164 | 0.235 | 24.7 |
| Comparative | DNA | perylene | 456,484 | 4.45 | 3.62 | 0.160 | 0.200 | 22.3 |
| Example 1 | | | | | | | | |

As shown in Table 1, using as a reference “Electroluminescent Efficiency/Y” value which exhibits a tendency similar to the quantum efficiency, when comparing the Comparative Example 1, which is an OLED device including a conventional well known electroluminescent material DNA:perylene, with the OLED device using organic electroluminescent compounds according to the present invention, the OLED device using organic electroluminescent compounds according to the present invention was exhibited the higher “Electroluminescent Efficiency/Y” value.

### INDUSTRIAL APPLICABILITY

In accordance with the organic electroluminescent compounds of the present invention, there is an advantage that an OLED device with a much superior driving life can be manufactured as it has a good electroluminescent efficiency and a superior life property of material. The organic electroluminescent compound of the present invention also is characterized by upgradedexcellent EL property when it is used as other layers as well as luminescent layer.

Those skilled in the art will appreciate that the conceptions and specific embodiments disclosed in the foregoing description may be readily utilized as a basis for modifying or designing other embodiments for carrying out the same purposes of the present invention. Those skilled in the art will also appreciate that such equivalent embodiments do not depart from the spirit and scope of the invention as set forth in the appended claims.

1. An organic electroluminescent compound represented by formula 1: 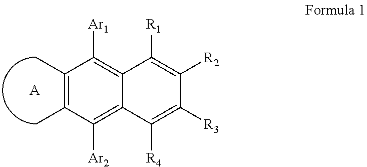
wherein, a ring A is a fused aryl group in which at least two rings are fused;
Arₗ and Ar₂ are independently a C₆-C₃₀ aryl group;
R₁ to R₄ are independently a hydrogen, a C₁-C₂₀ straight or branched chain alkyl group or alkoxy group and a C₆-C₃₀ aryl or heteroaryl group and a halogen group; and
the fused aryl group, the aryl group, heteroaryl group, the alkyl group and the alkoxy group are optionally substituted by a C₁-C₂₀ straight or branched chain alkyl group, a aryl group and halogen group.

2. The organic electroluminescent compound as set forth in claim 1, which is selected from formula 2 to formula 7: 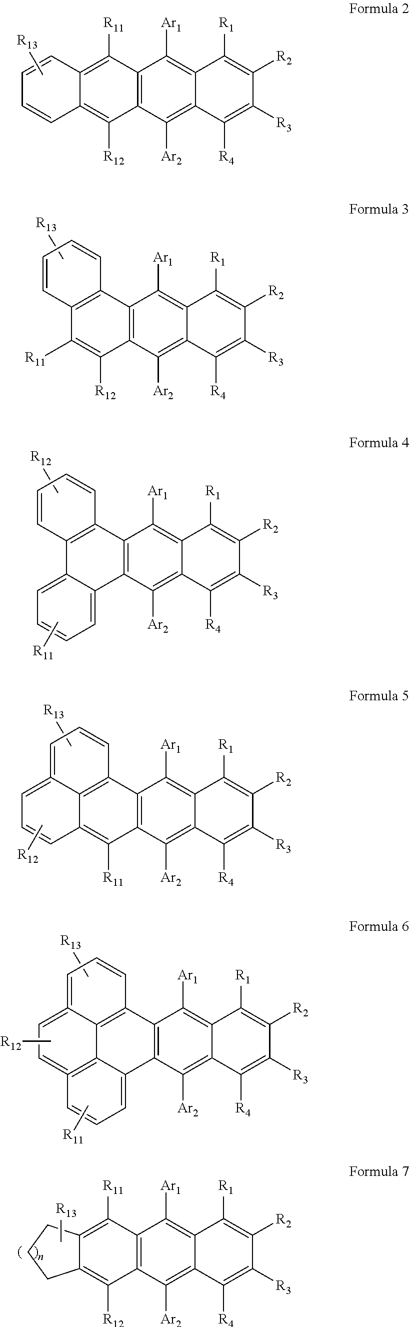
wherein, in the formula 2 to formula 7, Ar₁, Ar₂, R₁, R₂, R₃ and R₄ are the same as those defined in the formula 1 and R₁₁ to R₁₃ are independently a hydrogen, a C₁-C₂₀ straight or branched chain alkyl group or alkoxy group and a C₆⁻C₃₀ aryl or heteroaryl group and a halogen group; n is 1 to 3; and the alkyl group and the alkoxy group, the aryl group and heteroaryl group are optionally substituted by a C₁-C₂₀ straight or branched chain alkyl group, a aryl group and halogen group.

3. The organic electroluminescent compound as set forth in claim 1, wherein the Ar₁ and Ar₂ are independently selected from phenyl, tolyl, biphenyl, naphthyl, anthryl and fluorenyl.

4. The organic electroluminescent compound as set forth in claim 2, wherein the R₁ to R₄ and R₁₁ to R₁₃ are independently selected from hydrogen, methyl, ethyl, propyl, isopropyl, butyl, isobutyl, pentyl, hexyl, ethylhexyl, heptyl, octyl, isooctyl, nonyl, decyl, dodecyl, hexadecyl, cyclopentyl, cyclohexyl, phenyl, tolyl, byphenyl, benzyl, naphthyl, anthryl and fluorenyl.

5. The organic electroluminescent compound as set forth in claim 1, which is selected from following compounds:

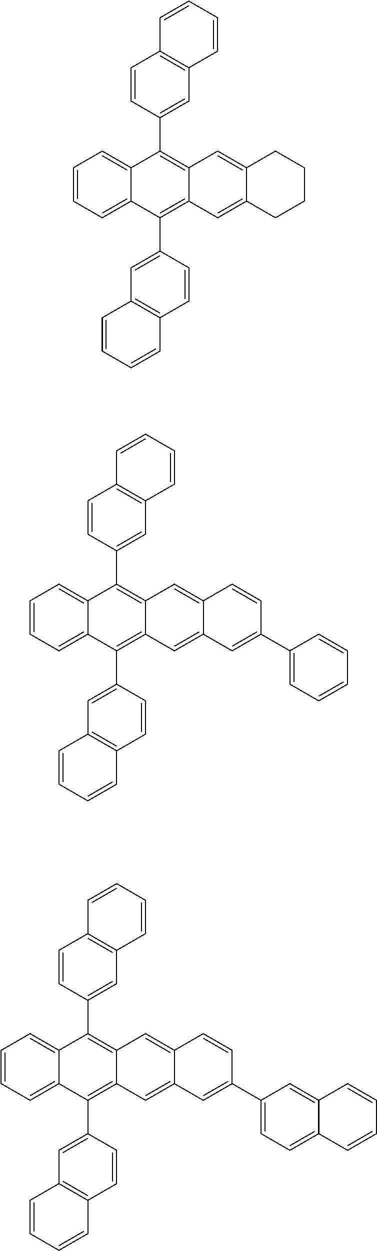

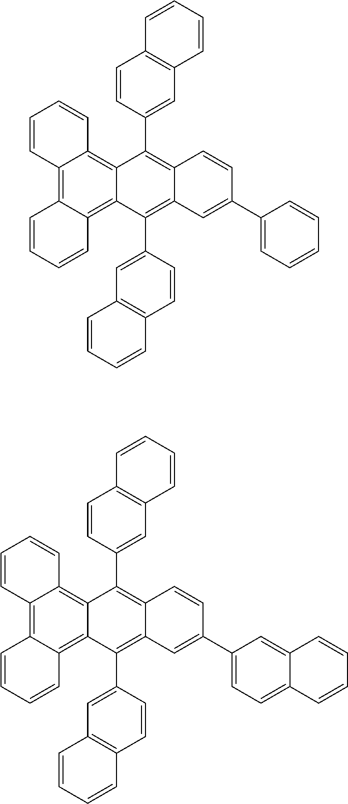

6. An electroluminescent device comprising the organic electroluminescent compound according to any one of claims 1 to 5.

7. An electroluminescent device containing the organic electroluminescent compounds between cathode and anode according to any one of claims 1 to 5.
